(19) United States
(12) Patent Application Publication
(10) Pub. No.: US 2012/0226322 A1
(43) Pub. Date: Sep. 6, 2012

(54) EXTRA-MEDULLARY CORTICAL BUTTRESS FIXATION DEVICE AND METHOD OF USE ASSOCIATED THEREWITH

(76) Inventor: Eduardo Gonzalez-Hernandez, Coconut Grove, FL (US)

(21) Appl. No.: 13/411,100

(22) Filed: Mar. 2, 2012

Related U.S. Application Data
(60) Provisional application No. 61/449,155, filed on Mar. 4, 2011.

(51) Int. Cl. A61B 17/80; A61B 17/88

(52) U.S. Cl. 606/286; 606/281

(57) ABSTRACT

Fixation devices serve in facilitating reduction and repair of a fractured humerus. In doing so, the fixation devices serve in maintaining the position of a bone plate relative to a fractured bone. The fixation devises include an extra-medullary component, and each of the bone fixation devices can buttress the cortical bone of the fractured bone.

The present application claims the benefit of provisional Application No. 61/449,155, filed Mar. 4, 2011; which is incorporated by reference herein.

### BACKGROUND OF THE INVENTION

1. Field of the Invention

The present invention generally relates to a bone plate and fixation device used to facilitate repair and fixation of a fractured bone. More particularly, the present invention relates to a bone plate and fixation device used to counteract deformation forces. More specifically, the present invention relates to a fixation device that maintains the connection of a bone plate with the portions of a fractured bone.

2. Description of the Prior Art

Fractures of bones such as the clavicle can be difficult to repair. For example, portions of a fractured clavicle are pulled in opposing directions by the interaction thereof with surrounding tissue. To illustrate, if the distal portion of a clavicle 10 is fractured as depicted in FIG. 1, the fractured clavicle 10 is subjected to deforming forces applied in at least two directions by the surrounding tissue.

The first direction (indicated by a first arrow F₁) of deforming force is applied to a first bone portion 12 of the fractured clavicle 10, and the second direction (indicated by a second arrow F₂) of deforming force is applied to a second bone portion 14 of the fractured clavicle 10. The first arrow F₁ corresponds to deforming forces from muscle traction superiorly, and the second arrow F₂ corresponds to deforming forces from muscle traction inferiorly.

Fixation of the fractured clavicle 10 previously has been accomplished using a plate 16 and bone screws 18 depicted in FIGS. 2 and 3. As depicted in FIG. 3, the bone screws 18 are inserted through apertures 20 formed in the plate 16 and into the fractured clavicle 10. The bone screws 18 serve to secure attachment of the first and second bone portions 12 and 14 to the plate 16, and the plate 16 serves to stabilize the fractured clavicle 10. In doing so, the bone screws 18 can be inserted through the near cortex bone 22, the far cortex bone 24, and the medullary canal 26 of the first and second portions 12 and 14.

However, the near cortex bone 22 and the far cortex bone 24 (of the first and second bone portions 12 and 14) are relatively thin, and thus, the threads of the bone screws 18 have a limited amount of bone to engage. That is, the relatively thin near and far cortex bones 22 and 24 afford limited purchase for the bone screws 18 therein. Accordingly, the deforming forces indicated by the arrows F1 and F2 can work to destroy the connection of the bone plate 16 with the first and second bone portions 12 and 14 afforded by the bone screws 18. Thus, the limited amount of engagement of the bone screws 18 afforded with the near and far cortex bone 22 and 24 can cause failure of the connection between the bone plate 16 and the first and second bone portions 12 and 14.

Therefore, there is a need for a fixation device and method of use associated therewith that is configured to counteract the above-discussed deformation forces and maintain the connection of a bone plate with the portions of a fractured bone.

### SUMMARY OF THE INVENTION

The present invention in a preferred embodiment contemplates an assembly for facilitating reduction and repair of a fractured bone, the assembly including a bone plate adapted to overlie and contact at least two bone portions of the fractured bone, the bone plate having a first end, a second end, a length between the first and second ends, a first surface adapted to contact the at least two bone portions, and a second surface opposite the first surface, the bone plate including a plurality of apertures formed therein, the plurality of apertures extending between the first and second surfaces thereof, at least a first aperture of the plurality of apertures being adapted to overlie a first bone portion of the at least two bone portions; and at least one fixation device adapted to secure the bone plate to the fractured bone, the at least one fixation device including at least one fastener and a buttress pin, each of the at least one fastener having a head portion, a shaft portion, and a hp portion with an aperture formed therein, a first fastener of the at least one fastener being received through the first aperture of the bone plate and through a first hole formed through the first bone portion, an entrance to the first hole being provided in a first surface of the first bone portion adjacent the bone plate, and an exit to the first hole being provided in a second surface of the first bone portion away from the bone plate, where, when the tip portion of the first fastener protrudes from the exit to the first hole, the buttress pin is received in the aperture of the tip portion, the first bone portion being positioned between the bone plate and the buttress pin.

The present invention in a further preferred embodiment contemplates a method for maintaining a connection between a bone plate and a fractured bone, the method including the acts of positioning the bone plate with respect to a first bone portion of the fractured bone; inserting a first fastener through a first aperture of a plurality of apertures formed in the bone plate and through a corresponding first aperture formed through the first bone portion of the fractured bone, where the first aperture in the first bone portion includes an entrance provided in a first surface of the first bone portion adjacent the bone plate, and an exit provided in a second surface of the first bone portion away from the bone plate; extending a tip portion of the first fastener out of the exit to the first aperture in the first bone portion; and inserting a buttress pin into an aperture formed in the tip portion of the first fastener, where the fractured bone is maintained between the bone plate and the buttress pin, and the buttress pin prevents withdrawal of the first fastener from the first aperture in the first bone portion.

The present invention in a yet a further preferred embodiment contemplates an assembly facilitating reduction and repair of a fractured bone, the assembly including a bone plate adapted to overlie and contact at least two bone portions of the fractured bone, the bone plate having a first end, a second end, a length between the first and second ends, a first surface adapted to contact the at least two bone portions, and a second surface opposite the first surface, the bone plate including a plurality of apertures formed therein, the plurality of apertures extending between the first and second surfaces thereof, at least a first aperture of the plurality of apertures being adapted to overlie a first bone portion of the at least two bone portions; and at least one fixation device adapted to secure the bone plate to the fractured bone, the at least one fixation device including at least one fastener and at least one washer adapted to engage the at least one fastener, each of the at least one fastener having a head portion, a shaft portion, and a tip portion, a first fastener of the at least one fastener being received through the first aperture of the bone plate and through a first hole formed through the first bone portion, an entrance to the first hole being provided in a first surface of the first bone portion adjacent the bone plate, and an exit to the first hole being provided in a second surface of the first bone portion away from the bone plate, where, when the tip portion protrudes from the exit to the first hole, a first washer of the at least one washer engages at least one of the shaft portion and the tip portion of the first fastener, a first surface of the first washer contacts the second surface of the first bone portion, and the first washer prevents withdrawal of the first fastener from the first hole.

it is understood that both the foregoing general description and the following detailed description are exemplary and exemplary only, and are not restrictive of the invention as claimed.

### BRIEF DESCRIPTION OF THE DRAWINGS

The accompanying drawings, which are incorporated in and constitute a part of the specification, illustrate preferred embodiments of the invention. Together with the description, they serve to explain the objects, advantages and principles of the invention. In the drawings:

FIG. 1 is a perspective view of a fractured bone having a first bone portion and second bone portion;

FIG. 2 is a perspective view of the first and second bone portions of the fractured bone of FIG. 1 maintained in position with respect to one another using a bone plate and bone screws;

FIG. 3 is a cross-sectional view of the bone plate and fractured bone depicted in FIG. 2 showing the bone screws received through the bone plate, and the first and second portions of the fractured bone;

FIG. 4 is a side devotional view of an embodiment of a fixation device according to the present invention in position relative to a fractured bone and a bone plate, of which the fractured bone and the bone plate are both shown in longitudinal cross section, the fixation device serving to maintain the position of the bone plate with respect to the fractured bone;

FIG. 5 is a bottom perspective view of the fixation device of FIG. 4 on the exterior of a portion of a fractured bone;

FIG. 6 is a front elevational view of the fixation device depicted in FIGS. 4 and 5 in position relative to the fractured bone (FIGS. 4 and 5) and the bone plate (FIG. 4), which are both shown in transverse cross section;

FIG. 7A is a perspective view of a first alternative version of a buttress pin for use with the fixation device of FIGS. 4-6;

FIG. 7B is a perspective view of a second alternative version of a buttress pin for use with the fixation device of FIGS. 4-6;

FIG. 8A is a perspective view of a third alternative version of a buttress pin for use with the fixation device of FIGS. 4-6;

FIG. 8B is a perspective view of a fourth alternative version of a buttress pin for use with the fixation device of FIGS. 4-6;

FIG. 9 is a side perspective view of another embodiment of a fixation device according to the present invention in position relative to a fractured bone, which is depicted in phantom;

FIG. 10 is a bottom perspective view of the fixation device of FIG. 9 on the exterior of a portion of a fractured bone;

FIG. 11A is a bottom perspective view of a fastener and a buttress pin used in the fixation devices depicted in FIGS. 4-6, 9, and 10, where a washer is received on the fastener and is positioned above the buttress pin;

FIG. 11B is a top plan view of the washer depicted in FIG. 11A;

FIG. 11C is a top plan view of a first alternative version of a washer;

FIG. 11D is a top plan view of a second alternative version of a washer;

FIG. 11E is a top plan view of a third alternative version of a washer;

FIG. 11F is a top plan view of a fourth alternative version of a washer;

FIG. 12 is a bottom perspective view of the washer depicted in FIG. 11C including an anti-expulsion feature included thereon;

FIG. 13A is a first perspective view of the installation of the washer depicted in FIGS. 11 and 12 with respect to a fractured bone and a tip portion of the fasteners depicted in FIGS. 4-6, 9, and 10;

FIG. 13B is a second perspective view of the installation of the washer depicted in FIGS. 11C and 12;

FIG. 13C is a third perspective view of the installation of the washer depicted in FIGS. 11C and 12;

FIG. 14 is a bottom perspective view of a fifth alternative version of a washer;

FIG. 15A is an exploded partially fragmentary perspective view of another embodiment of a fixation device according to the present invention;

FIG. 15B is a side partially fragmentary cross-sectional view of the fixation device of FIG. 15A positioned with respect to a fractured bone;

FIG. 16 is an exploded partially fragmentary perspective view of another embodiment of a fixation device according to the present invention;

FIG. 17A is a top perspective view of a sixth alternative view of a washer;

FIG. 17B is a side partially fragmentary cross-sectional view of another embodiment of the fixation device according to the present invention using the washer depicted in FIG. 17A;

FIG. 18A is a side perspective view of another embodiment of a fixation device according to the present invention;

FIG. 18B is a side partially fragmentary cross-sectional view of the fixation device of FIG. 18A positioned with respect to a fractured bone;

FIG. 19 is a bottom partially fragmentary perspective view of an articulable washer used with the fastener of the fixation devices depicted in FIGS. 4-6, 9, and 10;

FIG. 20A is a side partially fragmentary perspective view of the articulable washer of FIG. 19 and the fastener associated therewith showing a portion thereof in cross section, and depicting deformation forces applicable thereto;

FIG. 20B is a side partially fragmentary perspective cross-sectional view of a buttress pin and the fastener used with the articulable washer of FIG. 19 illustrating the potential effects of the deformation forces;

FIG. 21A is a fragmentary front elevational view of a first wire used with another embodiment of the fixation device according to the present invention;

FIG. 21B is a fragmentary front elevational view of a second wire used with another embodiment of the fixation device according to the present invention;

FIG. 22 is a side perspective view of another embodiment of a fixation device according to the present invention in position relative to a fractured bone and a bone plate, of which the fractured bone is shown in phantom, the fixation device serving to maintain the position of the bone plate with respect to the fractured bone;

FIG. 23 is a side perspective view of another embodiment of a fixation device according to the present invention in position relative to a fractured bone, of which the fractured bone is shown in phantom; and

FIG. 24 is a side perspective view of another embodiment of a fixation device according to the present invention.

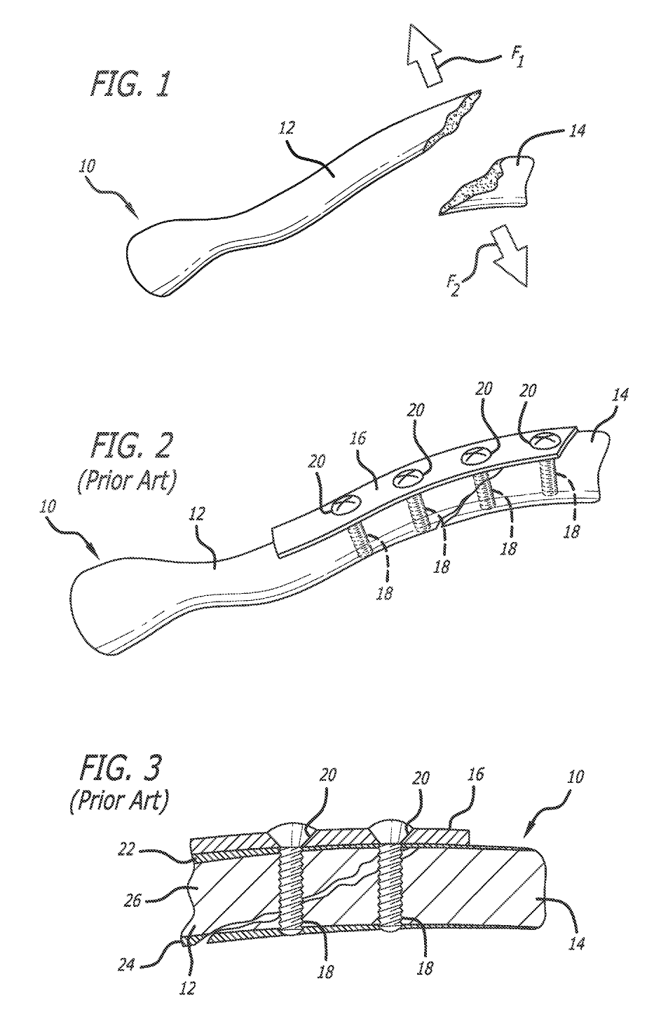

### DETAILED DESCRIPTION OF THE PREFERRED EMBODIMENT

The following description is intended to be representative only and not limiting, and many variations can be anticipated according to these teachings. Reference will now be made in detail to the preferred embodiments of this invention, examples of which are illustrated in the accompanying drawings.

Bone fixation devices of the present invention are discussed below being used in facilitating repair of fractured bones. Each of the fixation devices include an extra-medullary component serving to aid reduction and repair of fractured bones. As such, each of the bone fixation devices can serve in aiding reduction and repair of fractured bones by buttressing the cortical bone thereof. Furthermore, while various embodiments of the bone fixation devices are depicted being used with fractured clavicles, the present invention is not limited to use therewith. The bone fixation devices of the present invention can be used to repair other fractured bones.

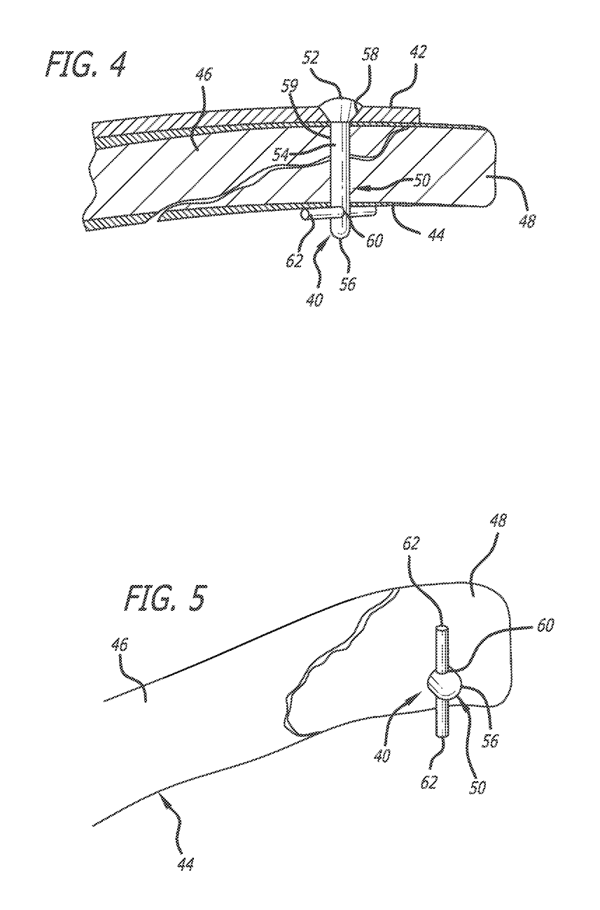

One embodiment of a fixation device of the present invention is generally indicated by the numeral 40 in FIGS. 4 and 5. FIG. 4 depicts a bone plate 42 and a fractured clavicle 44 (in cross-section) attached to one another using fixation device 40. Fractured clavicle 44 includes a first bone portion 46 and a second bone portion 48. Fixation device 40 is used in maintaining the connection between bone plate 42 and fractured clavicle 44. Furthermore, although not depicted in FIGS. 4 and 5, bone plate 42 can be attached to first and second bone portions 46 and 48 using bone (screws) inserted through apertures (not shown) formed in bone plate 42 and into fractured clavicle 44.

Fixation device 40 includes a fastener 50 having a head portion 52, a shaft portion 54, and a tip portion 56. During use thereof, fastener 50 is inserted through an aperture 58 formed in bone plate 42 into a corresponding aperture 59 formed in first and second bone portions 46 and 48. Fastener 50 can be a bone screw. As such, head portion 52 can be threaded to engage corresponding threads (not shown) formed in aperture 58. Furthermore, shaft portion 54 can be threaded to facilitate attachment to first and second bone portions 46 and 48. Shaft portion 54 can also be solid or hollow, and can be made of articulating components that allow shaft portion 54 to telescope (e.g., shorten or lengthen) to facilitate compression of bone plate 42 to fractured clavicle 44, and/or for the purpose of reduction or neutralization of fractured clavicle 44.

Tip portion 56 of fastener 50 includes an aperture 60 for receiving a buttress pin 62 therethrough. As depicted in FIGS. 4 and 5, when received in aperture 60, buttress pin 62 is disposed at an angle that is transverse (e.g., perpendicular or oblique) with respect to shaft portion 54. As such, the longitudinal axis of fastener 50 would be transverse to the longitudinal axis of buttress pin 62. Furthermore, to facilitate engagement with fastener 50, buttress pin 62 can be threaded to engage threads (not shown) provided in aperture 60.

The buttress pin 62 contacts fractured clavicle 44 and serves in maintaining the position of bone plate 42 with respect to fractured clavicle 44 to maintain the connection between bone plate 42 and fractured clavicle 44. That is, fractured clavicle 44 is entrapped between bone plate 42 and buttress pin 62, and thus, fixation device 40 serves to resist the above-discussed deforming forces. As such, buttress pin 62 strengthens the connection between fractured clavicle 44 and bone plate 42.

As depicted in FIGS. 4 and 5, the longitudinal axis of buttress pin 62 is disposed at an angle perpendicular to the longitudinal axis of fractured clavicle 44. However, the orientation of the longitudinal axis of buttress pin 62 is not limited to such a perpendicular angle. The longitudinal axis of buttress pin 62 can be disposed at a parallel angle with respect to the longitudinal axis of fractured clavicle 44 or provided at any transverse angle with respect to the longitudinal axis of the fractured clavicle.

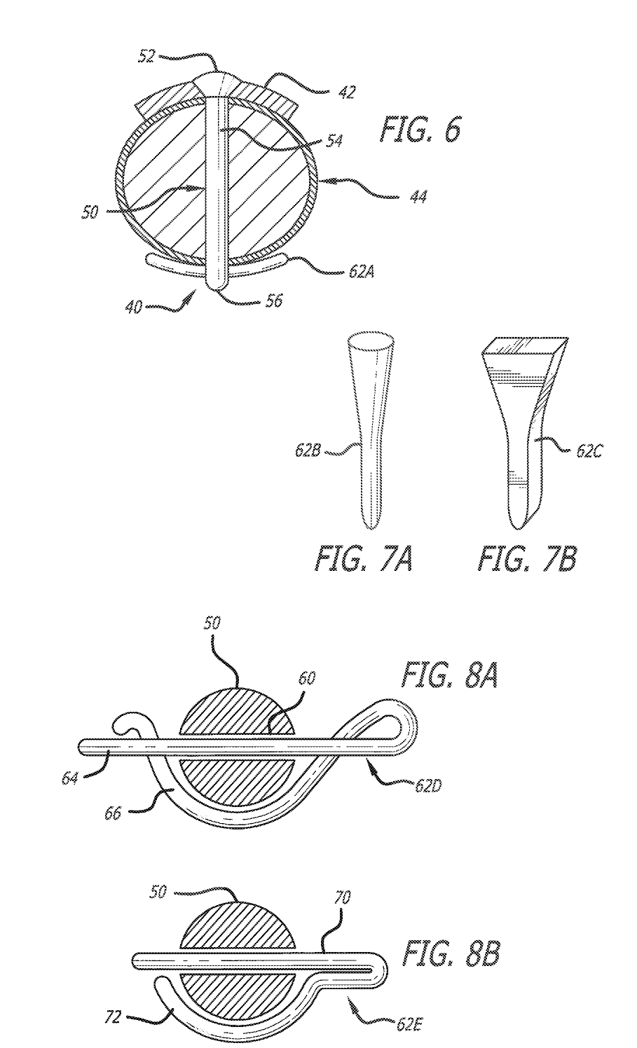

Buttress pin 62 depicted in FIGS. 4 and 5 is straight along its length. However, as depicted in FIG. 6, a buttress pin 62A can have a curvature along its length. The radius curvature of buttress pin 62A depicted in FIG. 6 can approximate (or be matched to) the curvature of a surface of fractured clavicle 44 to which buttress pin 62A is contacted. Furthermore, buttress pins 62 (FIGS. 4 and 5) and 62A (FIG. 6) can have arcuate (e.g., circular or ovoid) cross sections along their lengths. The cross section of buttress pin 62 can also vary in size and shape along its length. For example, as depicted in FIG. 7A, a buttress pin 62B has a varying cross section and is substantially conical, and, as depicted in FIG. 7B, a buttress pin 62C has a varying cross section and is substantially pyramidal. The varied cross sections thereof allow the buttress pins to be wedged into aperture 60 to prevent removal therefrom.

Furthermore, buttress pins 62D and 62E depicted in FIGS. 8A and 8B can be provided. As depicted in FIG. 8A, buttress pin 62D includes a first arm 64 and a second arm 66 used in clamping to fastener 50. First and second arms 64 and 66 are biased to at least in part overlap one another to facilitate such clamping action. During use, first arm 64 is received through aperture 60 of fastener 50, and second arm 66 is wrapped around the exterior of fastener 50. By wrapping around a portion of the exterior of fastener 50, second arm 66 inhibits first arm 64 from moving within aperture 60 and prevents buttress pin 62D from dislodging from fastener 50.

Like buttress pin 62D, buttress pin 62E, as depicted in FIG. 8B, includes a first arm 70 and a second arm 72. First and second arms 70 and 72 are biased in the position depicted in FIG. 8B, but first and second arms 70 and 72 do not overlap one another. During use, first arm 70 is received through aperture 60 of fastener 50, and second arm 72 is wrapped around the exterior of fastener 50. By wrapping around a portion of the exterior of fastener 50, second arm 72 inhibits first arm 70 from moving with the aperture and prevents buttress pin 62E from dislodging from fastener 50.

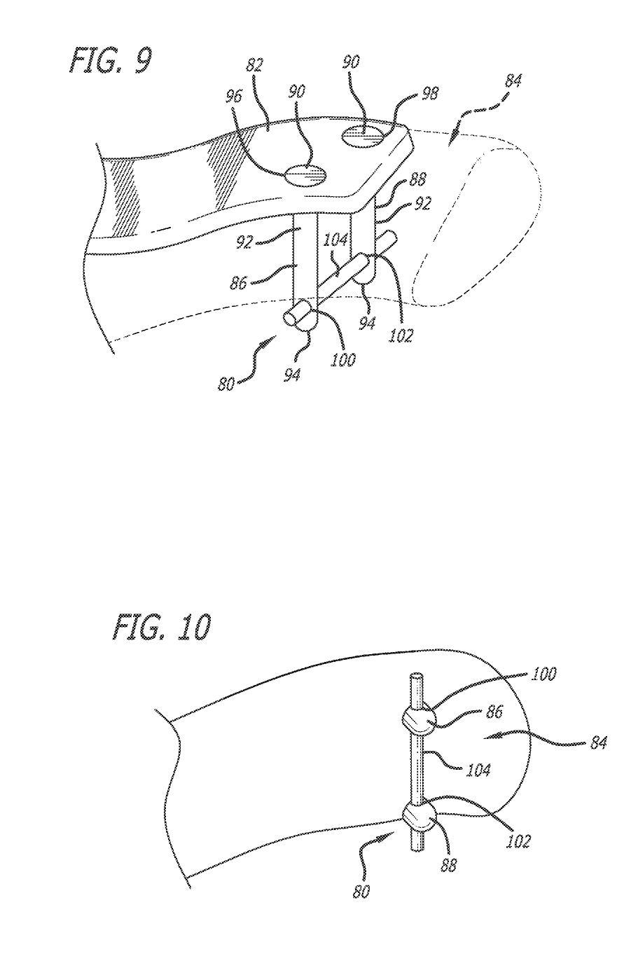

Yet another embodiment of a fixation device of the present invention is generally indicated by numeral 80 in FIGS. 9 and 10. FIG. 9 depicts a bone plate 82 and a fractured clavicle 84 (in phantom) attached to one another using fixation device 80. Fixation device 80 is used in maintaining the connection between bone plate 82 and fractured clavicle 84. However, rather than inserting a single fastener through fractured clavicle 84, fixation device 80 includes a first fastener 86 and a second fastener 88 to attach bone plate 82 to fractured clavicle 84.

Although not depicted in FIGS. 9 and 10, fractured clavicle 84 can include a first portion and a second portion joined to one another using bone plate 82 and fixation device 80. In addition to use of fixation device 80, bone plate 82 can be attached to the first and second bone portions of fractured clavicle 84 using bone screws (not shown) inserted through apertures (not shown) formed in bone plate 82 and into fractured clavicle 84.

Like fastener 50, first and second fasteners 86 and 88 have head portions 90, shaft portions 92, and tip portions 94. During use thereof, first and second fasteners 86 and 88 are inserted through apertures 96 and 98 formed in bone plate 82 and through corresponding apertures in fracture clavicle 84. First and second fasteners 86 and 88 can be received through apertures provided in same bone portion or different bone portions of fractured clavicle 82. Furthermore, head portions 90 can be threaded to engage corresponding threads (not shown) formed in apertures 96 and 98. Shaft portions 92 can be threaded to facilitate engagement with the apertures of and attachment to fractured clavicle 84.

Tip portions 94 of first and second fasteners 86 and 88 include apertures 100 and 102 for receiving a buttress pin 104 therethrough. As depicted in FIGS. 9 and 10, when buttress pin 104 is received in apertures 100 and 102, buttress pin 104 is disposed at an angle that is transverse (e.g. perpendicular or oblique) with respect to shaft portions 92. Furthermore, to facilitate engagement with first and second fasteners 86 and 88, buttress pin 104 can be threaded to engage threads (not shown) provided in apertures 100 and 102.

Buttress pin 104, like buttress pins 62 and 62A-E, serves in maintaining the position of bone plate 82 with respect to fractured clavicle 84. Furthermore, while buttress pin 104 depicted in FIGS. 9 and 10 is straight with an arcuate (e.g., circular or ovoid) cross section along its length, buttress pin 104 (like the variations of buttress pin 62 (i.e., buttress pins 62A, 62B, and 62C) can be curved with varying cross sections along its length. Furthermore, rather than using buttress pin 104 which engages each of first and second fasteners 86 and 88, fasteners 86 and 88 each can be configured to engage one of buttress pins 62 and 62A-62E.

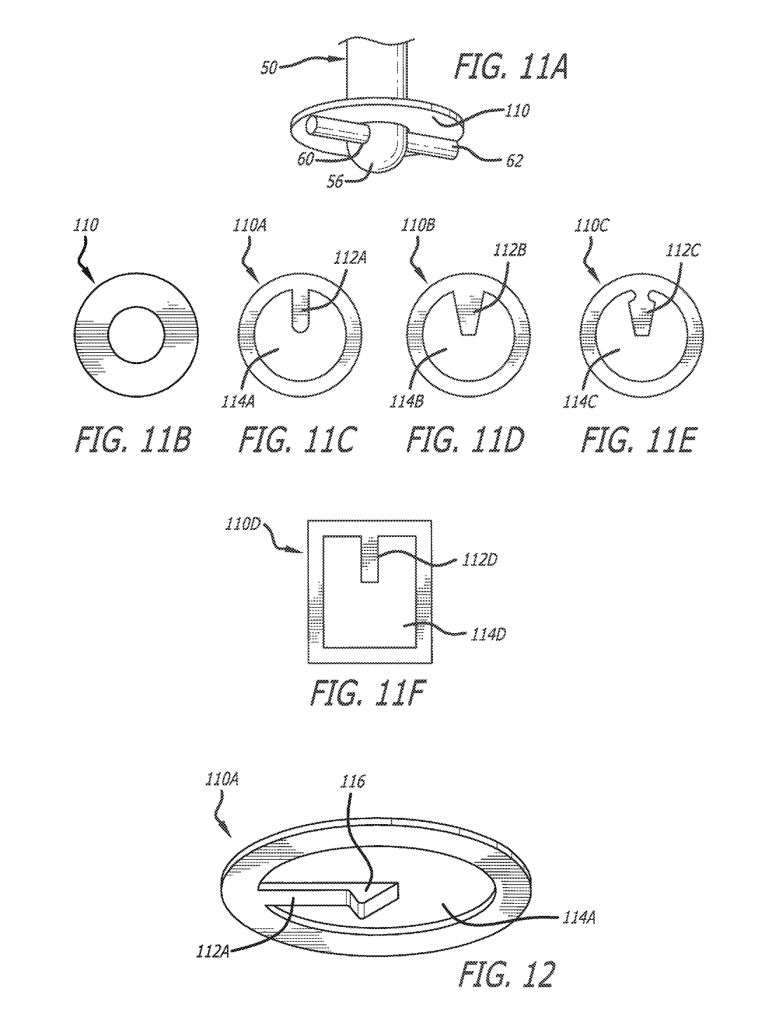

To aid the function of buttress pins 62, 62A-E, and 104, washers (such as a washer 110 depicted in FIGS. 11A and 11B) can be provided over fasteners 50, 86, and 88. For example, as depicted in FIG. 11A, a washer 110 is provided on fastener 50 above buttress pin 62 of fixation device 40. Thus, when fixation device 40 is applied to fractured clavicle 44, washer 110 is provided between buttress pin 62 and fractured clavicle 44. As such, washer 110 provides an additional surface to secure the attachment of bone plate 42 to fractured clavicle 44. Similarly, washer 110 can be used with fixation device 80. To that end, a first washer 110 can be provided on fastener 86 above buttress pin 104 and a second washer 110 can be provided on fastener 88 above buttress pin 104 to provide additional surface to secure the attachment of bone plate 82 to fractured clavicle 84.

Washer 110 (FIGS. 11A and 11B) can be modified to have various shapes, sizes, and features. For example, the variations of washer 110 are labeled 110A-D in FIGS. 11C-11F. Thus, while washers 110A-110D in FIGS. 11C-11F are either circular and rectangular, the washers are not limited thereto.

Washers 110A-C are circular in shape, and include projections 112A-C, respectively, extending into central cavities 114A-C thereof. Furthermore, washer 110D is rectangular in shape, and includes a projection 112D extending into a central cavity 114D thereof. Central cavities 114A-D are sized to receive fasteners 50, 86, and 88 therethrough.

Projections 112A-D are configured to extend into apertures 60, 100, and 102. When received in apertures 60, 100, and 102, projections 112A-D serve to prevent rotation of washers 110A-D on or about fasteners 50, 86, and 88. Washers 110A-D including projections 112A-D can also be used in place of buttress pins 62, 62A-E, and 104. That is, rather than using washers 110A-D in combination with buttress pins 62, 62A-E, and 104, washers 110A-D can be used instead of buttress pins 62, 62A-E, and 104. As such, in fixation devices 40 and 80, projections 112A-D can serve the function of buttress pins 62, 62A-E, and 104, and in doing so, maintain the positions of bone plates 42 and 82 with respect to fractured clavicles 44 and 84, respectively.

Projections 112A-D can be shaped to wedge against the interiors of apertures 60, 100, and 102 to resist withdrawal of projections 112A-D therefrom. As depicted in FIGS. 110 and 11E, projections 112B and 112C include such wedge shapes. Furthermore, projections 112A-D can be modified to be resilient and include an anti-expulsion feature to prevent withdrawal thereof from apertures 60, 100, and 102. For example, FIG. 12 depicts projection 112A of washer 110A including an anti-expulsion feature 116. Anti-expulsion feature 116 is formed as a wedge-shaped catch that ultimately prevents projections 112A from withdrawing from apertures 60, 100, and 102.

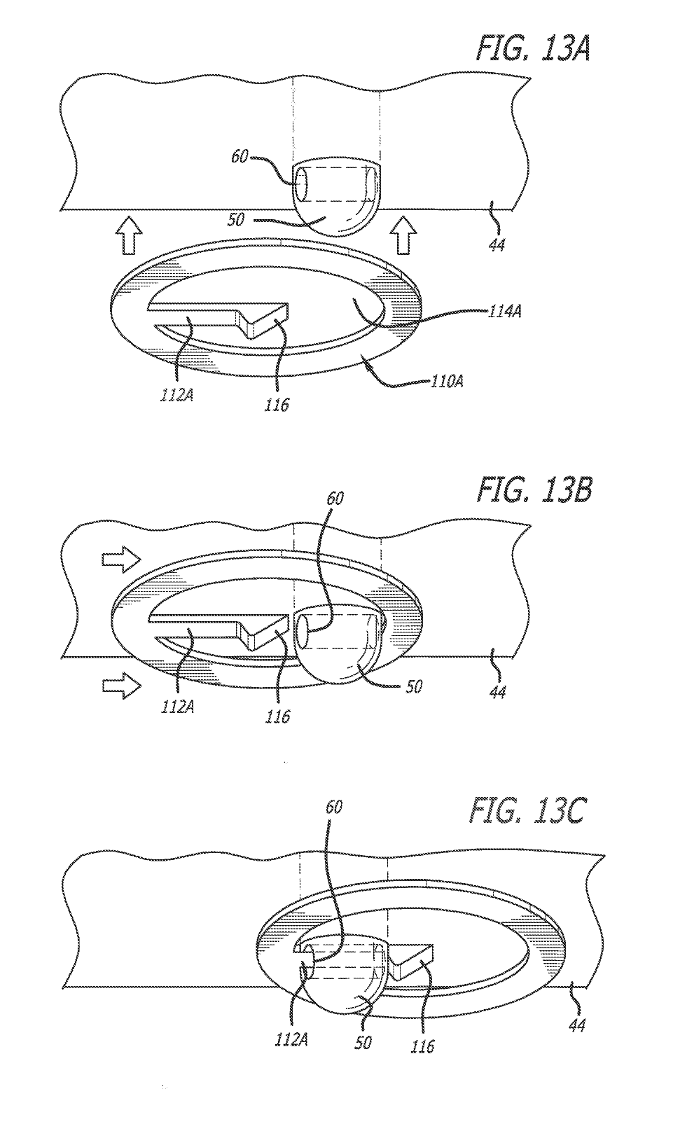

During use of washer 110A including projection 112A with anti-expulsion feature 116, washer 110A is initially positioned adjacent fastener 50 as depicted in FIG. 13A. Thereafter, as depicted in FIG. 13B, fastener 50 is inserted into central cavity 114A such that projection 112A is aligned with an entrance to aperture 60. Projection 112A is then inserted into the entrance to aperture 60, and, when anti-expulsion feature 116 advances through the exit to aperture 60, anti-expulsion feature 116 snaps into position to prevent withdrawal of projection 112A. In essence, as depicted in FIG. 13C, anti-expulsion feature 116 catches on the exterior of fastener 50 to prevent withdrawal of projection 112A from aperture 60.

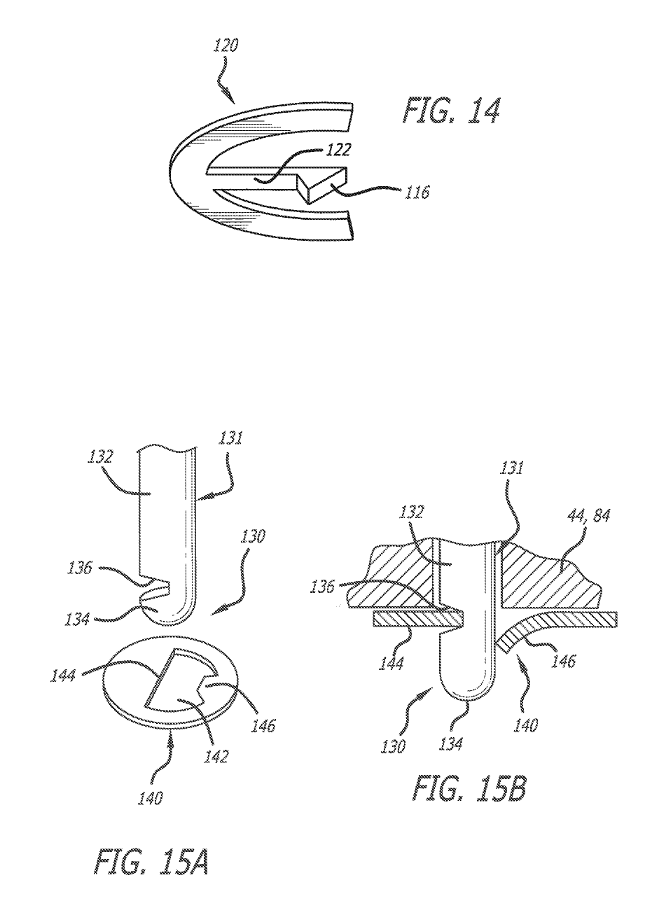

While depicted in FIGS. 12 and 13A-C as being provided on projection 112A, anti-expulsion feature 116 can also be provided on projections 112B-D. Furthermore, washers 110A-D can be provided with incomplete perimeters. For example, FIG. 14 depicts a washer 120 having an incomplete perimeter with a projection 122 having anti-expulsion feature 116.

As depicted in FIGS. 15A-18B, additional embodiments of fixation devices according to the present invention are generally indicated by the numerals 130, 150, 170, and 190. The fixation devices 130, 150, 170, and 190 include additional configurations of fasteners and corresponding washers. Like fasteners 50, 86, and 88, and washers 110, 110A-D, and 120, the fasteners and the corresponding washers depicted in FIGS. 15A-18B of fixation devices 130, 150, 170, and 190 serve in holding bone plates 42 and 82 in position with respect to fractured clavicles 44 and 84, respectively.

The fasteners depicted in FIGS. 15A-15B are inserted through bone plates (such as bone plates 42 and 82) and through corresponding apertures in fractured clavicles (such as fractured clavicles 44 and 84) and receive washers thereon. Like fasteners 50, 86, and 88, fasteners depicted in FIGS. 15A-18B can include threaded head portions (not shown) and threaded shaft portions (not shown) to engage the bone plates and the fractured clavicles, respectively. Furthermore, like washers 110, 110A-110D, and 120, the washers depicted in FIGS. 15A-18B are used in maintaining the position of the bone plates with respect to the fractured clavicles to facilitate repair thereof.

FIGS. 15A and 15B depict a fixation device 130 including a fastener 131 having a shaft portion 132 and a tip portion 134. Fixation device 130 also includes a washer 140. A mouth (e.g., an indentation or a notch) 136 is formed in tip portion 134 for receiving a portion of washer 140. Mouth 136 could also be formed in shaft portion 132 adjacent tip portion 134. Like washers 110 and 110A-D, washer 140 includes a cavity 142 for receiving fastener 131 therein. However, instead of including a projection (such as projections 112A-D), washer 140 includes a tongue 144 and a resilient detent 146 bordering cavity 142. As depicted in FIG. 15B, when washer 140 is engaged to fastener 131, tongue 144 is received in mouth 136, and resilient detent 146 is deformed against shaft portion 132. Interaction of tongue 144 with mouth 136, and the force applied by resilient detent 146 against shaft portion 132 serve in resisting removal of washer 140 from fastener 131. In doing so, fixation device 130 can serve in maintaining the connection between a bone plate and a fractured clavicle to facilitate repair of the clavicle.

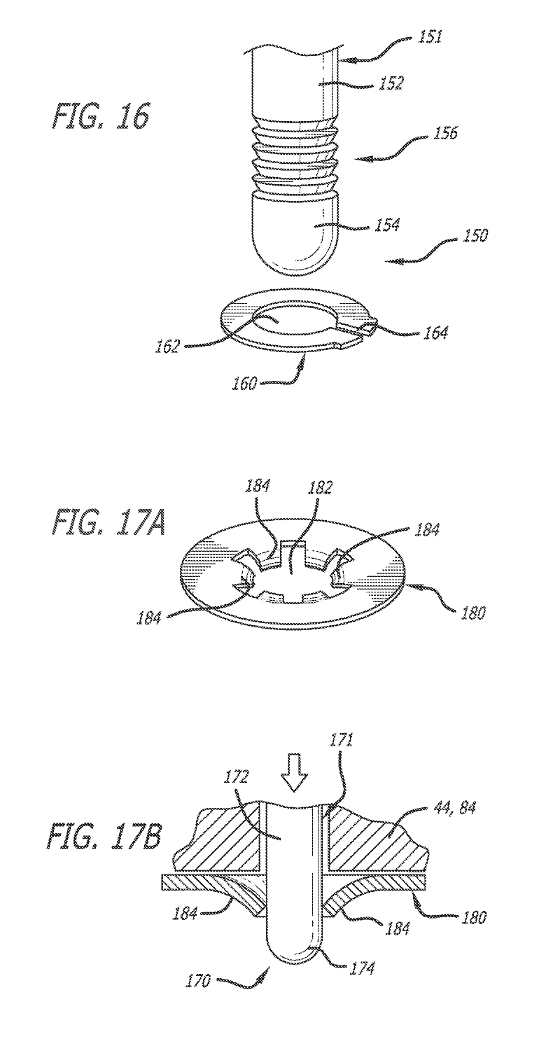

FIG. 16 depicts a fixation device 150 including a fastener 151 having a shaft portion 152 and a tip portion 154. Fixation device 150 also includes a spring washer 160. Ratchets 156 can be formed on fastener 151 along shaft portion 152 and tip portion 154. As depicted in FIG. 16, ratchets 156 extend from the tip portion 154 along the shaft portion 152 and/or tip portion 154. Ratchets 156 form ridges and valleys on fastener 151 that are configured to engage a spring washer 160. Spring washer 160 includes a central cavity 162 for receiving the fastener therein, and a break 164 formed therein. Break 164 allows central cavity 162 to expand and contract. Accordingly, when received over fastener 151, spring washer 160 can expand and contract to afford movement along ratchets 156. Furthermore, given the angle thereof, ratchets 156 resist removal of spring washer 160 from fastener 151. Ultimately, spring washer 160 is received in a valley formed by ratchets 156, and a ridge prevents spring washer 160 from being removed from fastener 151. In doing so, fixation device 150 can serve in maintaining the connection between a bone plate and a fractured clavicle to facilitate repair of the clavicle.

FIGS. 17A and 17B depict a fixation device 170 including a fastener 171 having a shaft portion 172 and a tip portion 174. Fixation device 170 also includes a washer 180. Shaft portion 172 and tip portion 174 can include surface roughenings (not shown) to resist removal of a washer 180 therefrom. Washer 180 includes a central cavity 182 for receiving fastener 171 therein, and resilient detents 184 extending into central cavity 182. When washer 180 is received on fastener 171, resilient detents 184 engage shaft portion 172 and/or tip portion 174 to resist removal of washer 180 from fastener 171. That is, the force applied by resilient detents 184 against shaft portion 172 and/or tip portion 174 generates friction (aided by the above-discussed surface roughenings) which serves in preventing removal of washer 180 from fastener 171. In doing so, fixation device 170 can serve in maintaining the connection between a bone plate and a fractured clavicle to facilitate repair of the clavicle.

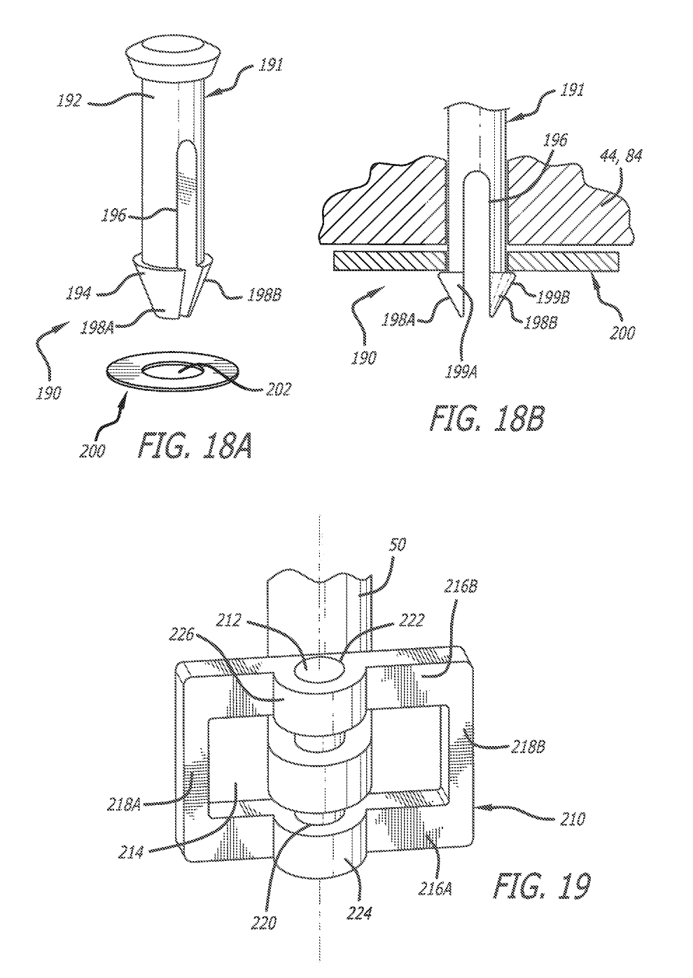

FIGS. 18A and 186 depict a fixation device 190 including a fastener 191 having a shaft portion 192, a tip portion 194, and channel 196 dividing tip portion 194 and a portion of shaft portion 192. Fixation device 190 also includes a washer 200. Tip portion 194 includes a first flared portion 198A and a second flared portion 198B provided on opposite skies of channel 196. Channel 196 permits deflection of first and second flared portions 198A and 198B towards one another, and first and second flared portions 198A and 198B include ramped surfaces 199A and 199B, respectively, for engaging a washer 200. Washer 200 includes a central cavity 202 for receiving fastener 191 therein. When received in central cavity 202, engagement of washer 200 with ramped surfaces 199A and 199B deflects first and second flared portions 198A and 198B towards one another to avow passage of washer 200 thereby. That is, upon initial insertion of fastener 191 into central cavity 202 and contact with washer 200, first and second flared portions 198A and 198B deflect towards one another from their original position (FIG. 18A), and thereafter, return to their original position when washer 200 passes thereby. Thereafter, as depicted in FIG. 18B, first and second flared portions 198A and 198B prevent removal of washer 200 from fastener 191. In doing so, fixation device 190 can serve in maintaining the connection between a bone plate and a fractured clavicle to facilitate repair of the clavicle.

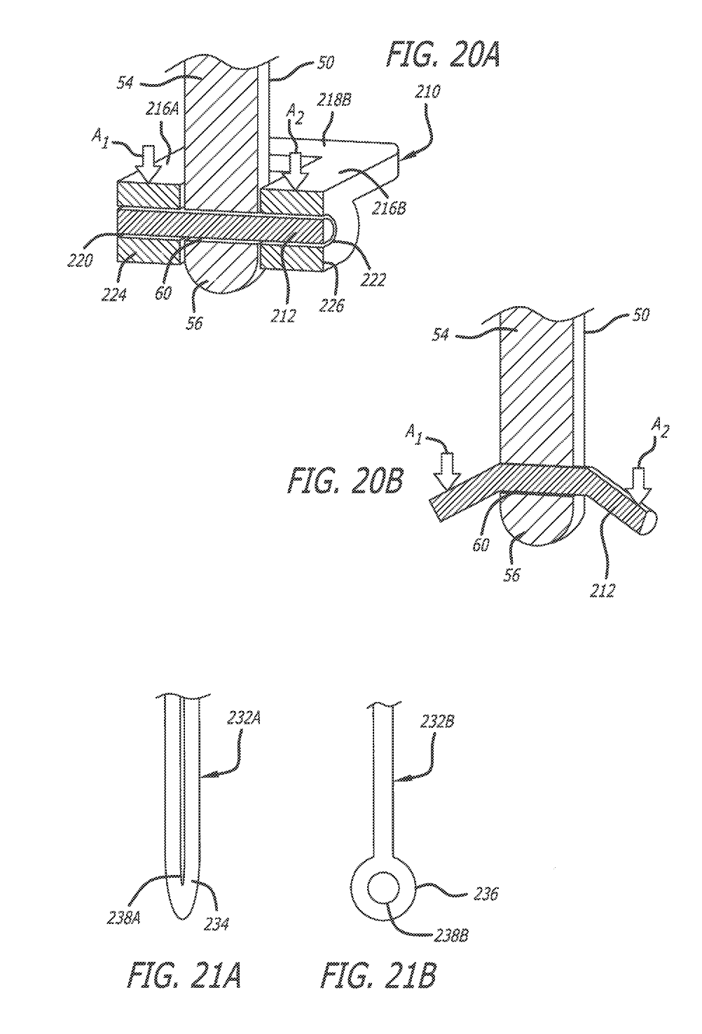

As depicted in FIGS. 19 and 20A, an articulatable washer 210 (FIGS. 19 and 20A) can be provided to engage fasteners 50, 86, and 88 in fixation systems 40 and 80. As depicted in FIGS. 19 and 20A, for example, articulatable washer 210 is used with fastener 50 of fixation system 40. Articulatable washer 210 is attached to fastener 50 using a buttress pin 212, and is capable of pivotable movement about buttress pin 212.

Articulable washer 210 can be rectangular in shape, and includes a central cavity 214 for receiving fastener 50 therein. Central cavity 214 is formed between first and second end portions 216A and 216B, and between third and fourth end portions 218A and 218B of articulatable washer 210. A first opening 220 and a second opening 222 are formed in articulatable washer 210 on either side of central cavity 212. First and second openings 220 and 222 are formed through built-up portions 224 and 226, respectively, formed on first and second end portions 216A and 216B. First and second openings 220 and 222 are sized to receive buttress pin 212.

Forces applied downwardly on third and fourth end portions 218A and 218B cause washer 210 to pivot about buttress pin 212. Furthermore, downward forces (designated by arrows A₁ and A₂) applied downwardly on first and second end portions 216A and 216B are resisted not only by buttress pin 212, but also by articulatable washer 210. Thus, articulatable washer 210 accommodates downward forces applied to third and fourth portions 218A and 218B by pivoting. Furthermore, washer 210 accommodates downward forces applied to first and second end portions 216A and 216B by preventing rotation movement with built-up portions 224 and 226, and by reinforcing buttress pin 212. Without the rotation prevention and reinforcement provided by articulatable washer 210, buttress pin 212 may succumb to the downward forces (designated by arrows A₁ and A₂) and deform (FIG. 20B).

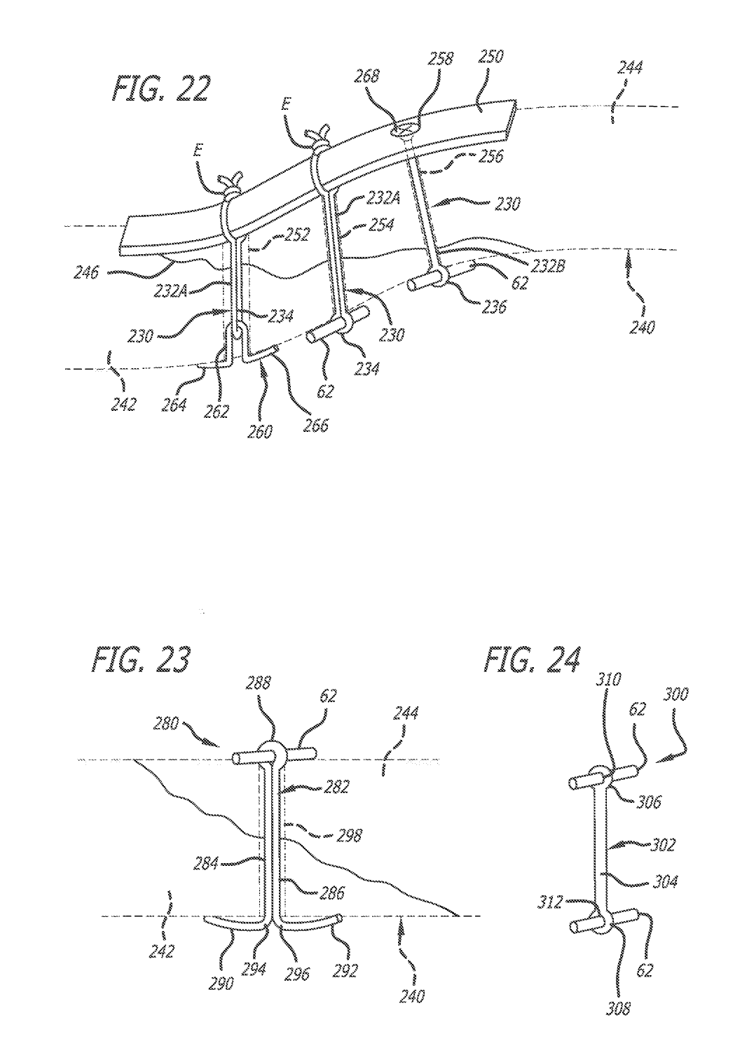

in another embodiment of a fixation device of the present invention is generally indicated by the numeral 230 in FIGS. 21A, 21B, and 22. In placement of the above-described fasteners, a wire (or equivalent such as a cable, a suture, etc.) is used. The wires used in fixation device 230 include a wire 232A (FIG. 21A) and 232B (FIG. 21B). Wire 232A can include a loop 234 and wire 232B can include an eyelet 236 formed thereon. Loop 234 and eyelet 236 includes aperture 238A and 238B, respectively. In fixation device 230, aperture 238A of loop 234 and aperture 238B of eyelet 236 can be configured to receive buttress pins (such as buttress pin 62) therein. Wire 232A and 232B (or the cable, the suture, etc.) has little (if any) intrinsic stiffness thereto, and thus, tension applied thereto is used to hold bone portions together and further buttress a fractured bone.

A fractured bone 240 is depicted in FIG. 22 in phantom, includes a first portion 242 and a second portion 244 divided by a fracture line 246. A bone plate 250 is provided to splint fractured bone 240, an aperture 258 is provided in bone plate 250, and three (3) holes 252, 254, and 256 are drilled through fractured bone 240 to receive wires 232A and 232B therethrough. As depicted in FIG. 22, aperture 258 of bone plate 250 corresponds to hole 256 formed in fractured bone 240. As discussed below, wires 232A and 232B (of fixation device 230) are used in addition to bone plate 250 to buttress fractured bone 240.

As depicted in FIG. 22, holes 252 and 254 each receive one of looped wires 232A such that bops 234 exit from fractured bone 240 opposite from bone plate 250. Thereafter, apertures 238A of bops 234 can receive buttress pins of various sizes and shapes. For example, buttress pin 260 is received through aperture 238A provided by loop 234 of wire 232A inserted into hole 252. Furthermore, buttress pin 62 (from fixation device 40) is received through aperture 238A provided by loop 234 of wire 232A inserted into hole 254. Thereafter, wires 232A (received in holes 262 and 264) are tensioned by tying ends E thereof opposite from loops 234 to bone plate 260. By tensioning wires 232A, buttress pin 62 is drawn toward fractured bone 240, and in doing so, the connection between plate 250 and fractured bone 240 is strengthened.

Buttress pin 260 is somewhat omega-shaped, and includes a body portion 262 and two leg portions 264 and 266. At least a portion of body portion 262 is received with hole 252 in fractured bone 240, and leg portions 264 and 266 contact fractured bone 240 around hole 252. As such, leg portions 264 and 266 prevent buttress pin 260 from being drawn in hole 252.

As depicted in FIG. 22, the wire 232B is inserted through aperture 258 into hole 256 such that eyelet 236 exits from fractured bone 240 opposite from bone plate 250. Thereafter, buttress pin 62 is received in aperture 238B of eyelet 236 of wire 232B, and the end of wire 232B opposite from eyelet 236 is fastened to bone plate 250 to tension wire 232B. To that end, the end of wire 232B can be pinned within aperture 258 using, for example, a set screw 268. To tension wire 232B (after buttress pin 62 is received in eyelet 236), wire 232B is drawn back through aperture 258, and then pinned to bone plate 250 using set screw 268. Excess portions of wire 232B can be removed once the remainder thereof is pinned to plate 250. Furthermore, while use of set screw 268 is used to facilitate attachment of wire 232B under tension, attachment of wire 232B is not limited thereto, and other attachment mechanisms can be used.

Rather than using a wire having little (if any) intrinsic stiffness, a rod 282 (having significant stiffness relative to wires 232A and 232B, but still being malleable) can be used in another embodiment of a fixation device generally indicated by the numeral 280 in FIG. 23. As depicted in FIG. 23, rod 282 can be used in joining first and second portions 242 and 244 (of fractured bone 240 together). Rod 282 can be used in place of or in addition to wires 232A and 232B depicted in FIG. 22, and fixation device 280 can be used in place of or with bone plate 250.

As depicted in FIG. 23, rod 282 includes a first body portion 284 and a second body portion 286 joined to one another at a loop portion 288. In addition to loop portion 288 portion at one end of first and second body portions 284 and 286, first and second arm portions 290 and 292 extend outwardly from first and second body portions 284 and 286, respectively, at the other end thereof. First and second arm portions 290 and 292 are joined to first and second body portions 284 and 286 at first and second bends 294 and 296, respectively.

During use, rod 282 is inserted into a hole 298 formed through first and second bone portions 242 and 244 such that loop 288 exits from fractured bone 240 opposite from first and second arms 290 and 292. Buttress pin 62 is then inserted into loop 288, and thus, first and second bone portions 242 and 244 are entrapped between buttress pin 62 and first and second arms 290 and 292. To provide additional tension in rod 282, the location of first and second bends 294 and 296 can be altered by deforming rod 282 such that first and second arm portions 290 and 292 are decreased in length, and first and second bends 294 and 296 are made to be closer to loop portion 288.

Additionally, a rod 302 (having significant stiffness relative to wires 232A and 232B, but still being malleable) can be used in another embodiment of the fixation device generally indicated by the numeral 300 in FIG. 24. Like rod 282, rod 302 can be used in joining first and second portions 242 and 244 (of fractured bone 240 together). Rod 302 can be used in place of or in addition to wires 232A and 232B depicted in FIG. 22 and rod 282 depicted in FIG. 23, and fixation device 300 can be used in place of or with bone plate 250.

Rod 302 includes a body portion 304, and eyelets 306 and 308 formed on either end of body portion 304. Eyelets 306 and 308 include apertures 310 and 312, respectively, formed therein. Apertures 310 and 312 can be configured to each receive a buttress pin (such as one of buttress pins 62) therethrough. Thus, when rod 302 is received in a hole formed through first and second bone portions (such as first and second bone portions 242 and 244), one of apertures 310 and 312 (of eyelets 306 and 308, respectively) would be provided at the entrance to the hole, and the other of apertures 310 and 312 would be provided at the exit to the hole. Thereafter, buttress pins 62 could be received in apertures 310 and 312 to entrap a fractured bone (such as fractured bone 240) therebetween.

Other embodiments of the invention will be apparent to those skilled in the art from consideration of the specification and practice of the invention disclosed herein. Accordingly, it is intended that the specification and examples be considered as exemplary only.

I claim:

1. An assembly for facilitating reduction and repair of a fractured bone, the assembly comprising:
a bone plate adapted to overlie and contact at least two bone portions of the fractured bone, said bone plate having a first end, a second end, a length between said first and second ends, a first surface adapted to contact the at least two bone portions, and a second surface opposite said first surface, said bone plate including a plurality of apertures formed therein, said plurality of apertures extending between said first and second surfaces thereof, at least a first aperture of said plurality of apertures being adapted to overlie a first bone portion of the at least two bone portions; and
at least one fixation device adapted to secure said bone plate to the fractured bone, said at least one fixation device including at least one fastener and a buttress pin, each of said at least one fastener having a head portion, shaft portion, and a tip portion with an aperture formed therein, a first fastener of said at least one fastener being received through said first aperture of said bone plate and through a first hole formed through the first bone portion, an entrance to the first hole being provided in a first surface of the first bone portion adjacent said bone plate, and an exit to the first hole being provided in a second surface of the first bone portion away from said bone plate, wherein, when said tip portion of said first fastener protrudes from the exit to the first hole, said buttress pin is received in said aperture of said tip portion, the first bone portion being positioned between said bone plate and said buttress pin.

2. The assembly of claim 1, wherein said buttress pin prevents withdrawal of said first fastener from said first hole.

3. The assembly of claim 1 wherein said buttress pin has a length and a cross section that varies along the length thereof, said varied cross section affording said buttress pin to be wedged into said aperture formed in said tip portion.

4. The assembly of claim 1, wherein said buttress pin has a length, said buttress pin being curved along the length thereof, the radius of curvature of said buttress pin approximating the curvature of the second surface of the first portion.

5. The assembly of claim 1, wherein the buttress pin includes a first arm and a second arm, said first arm being received in said aperture formed in said tip portion, and said second arm being wrapped around a portion of the exterior of said first fastener, said second arm preventing said buttress pin from dislodging from said first fastener.

6. The assembly of claim 1, further comprising a washer received on said first fastener, said washer being positioned between said buttress pin and the first bone portion.

7. The assembly of claim 6, wherein said washer includes a central cavity sized to receive said first fastener, and a projection extending into said central cavity, said projection being received in said aperture formed in said tip portion.

8. The assembly of claim 7, wherein said projection of said washer includes an anti-expulsion feature adapted to prevent withdrawal thereof from said aperture formed in said tip portion.

9. The assembly of claim 1, further comprising a washer, said washer including a central cavity sized to receive said first fastener and openings adjacent said central cavity for receiving said buttress pin, wherein, to attach said washer to said first fastener, said buttress pin is received through said aperture of said tip portion of said first fastener and said openings adjacent said central cavity of said washer, said washer being pivotable relative to said first fastener about said buttress pin.

10. The assembly of claim 1, further comprising a second aperture of said plurality of apertures formed in said bone plate, and a second fastener of said at least one fastener, said second aperture being adapted to overlie the first bone portion, said second fastener being received through said second aperture and through a second hole formed through the first bone portion, an entrance to the second hole being provided in the first surface of the first bone portion, and an exit to the second hole being provided in the second surface of the first bone portion, wherein, when said tip portion of said second fastener protrudes from the exit to said second hole, said buttress pin is received in said aperture of said tip portion.

11. The assembly of claim 10, further comprising a first washer received on said first fastener, and a second washer received on said second fastener, said first and second washer being positioned between said buttress pin and said first bone portion.

12. A method for maintaining a connection between a bone plate and a fractured bone, the method comprising:
positioning the bone plate with respect to a first bone portion of the fractured bone;
inserting a first fastener through a first aperture of a plurality of apertures formed in the bone plate and through a corresponding first aperture formed through the first bone portion of the fractured bone, wherein the first aperture in the first bone portion includes an entrance provided in a first surface of the first bone portion adjacent the bone plate, and an exit provided in a second surface of the first bone portion away from said bone plate;
extending a tip portion of the first fastener out of the exit to the first aperture in the first bone portion; and
inserting a buttress pin into an aperture formed in the tip portion of the first fastener, wherein the fractured bone is maintained between the bone plate and the buttress pin, and the buttress pin prevents withdrawal of the first fastener from the first aperture in the first bone portion.

13. The method of claim 12, further comprising positioning a first washer over the tip portion of the first fastener before the buttress pin is inserted into the aperture in the tip portion of the first fastener, the first washer contacting the second surface of the first bone portion.

14. The method of claim 12, further comprising:
inserting a second fastener through a second aperture of the plurality of aperture formed in the bone plate and through a corresponding second aperture formed through the second bone portion of the fractured bone, the second aperture extending between the first and second surfaces of the first bone portion;
extending a tip portion of the second fastener through an exit to the second aperture formed through the second bone portion; and
inserting the buttress pin into an aperture formed in the tip portion of the second fastener, the buttress pin engaging the apertures formed in the tip portions of both of the first and second fasteners, and the buttress pin preventing withdrawal of both of the first and second fasteners from the first and second apertures, respectively, formed in the first bone portion.

15. The method of claim 14, further comprising positioning a second washer over the tip portion of the second fastener before the buttress pin is inserted into the aperture in the tip portion of the second fastener, the second washer contacting the second surface of the first bone portion.

16. An assembly for facilitating reduction and repair of a fractured bone, the assembly comprising:
a bone plate adapted to overlie and contact at least two bone portions of the fractured bone, said bone plate having a first end, a second end, a length between said first and second ends, a first surface adapted to contact the at least two bone portions, and a second surface opposite said first surface, said bone plate including a plurality of apertures formed therein, said plurality of apertures extending between said first and second surfaces thereof, at least a first aperture of said plurality of apertures being adapted to overlie a first bone portion of the at least two bone portions; and
at least one fixation device adapted to secure said bone plate to the fractured bone, said at least one fixation device including at least one fastener and at least one washer adapted to engage said at least one fastener, each of said at least one fastener having a head portion, a shaft portion, and a tip portion, a first fastener of said at least one fastener being received through said first aperture of said bone plate and through a first hole formed through the first bone portion, an entrance to the first hole being provided in a first surface of the first bone portion adjacent said bone plate, and an exit to the first hole being provided in a second surface of the first bone portion away from said bone plate, wherein, when said tip portion protrudes from the exit to the first hole, a first washer of said at least one washer engages at least one of said shaft portion and said tip portion of said first fastener, a first surface of said first washer contacts the second surface of the first bone portion, and said first washer prevents withdrawal of said first fastener from said first hole.

17. The assembly of claim 16, wherein said one of said shaft portion and said tip portion of said first fastener includes an indentation formed therein, and said first washer includes a cavity, a tongue bordering said cavity, and a detent bordering said cavity, said tongue being received in said indentation, and said detent deforming against said one of said shaft portion and said tip when said first washer is engaged to said first fastener.

18. The assembly of claim 16, wherein of one of said shaft portion and said tip portion of said first fastener includes ratchets thereon, and said first washer is adapted to expand and contract over said ratchets, said ratchets affording movement of said first washer in a first direction, and preventing movement of said first washer in a second direction opposite said first direction when said first washer is engaged to said first fastener.

19. The assembly of claim 16, wherein said first washer includes a central cavity and at least three detents bordering said central cavity, said at least three detents deforming against one of said shaft portion and said tip portion of said first fastener to prevent movement of said first washer in one direction along said first fastener when said first washer is engaged to said first fastener.

20. The assembly of claim 16, wherein said first fastener includes a channel dividing said tip portion, said tip portion including a first flared portion and a second flared portion spaced apart from one another across said channel, said first and second flared portions allowing movement of said first washer in a first direction thereover via deflection towards one another across said channel, and said first and second flared portions preventing movement of said first washer in a second direction thereover when said first washer is engaged to said first fastener.
